(19) United States
(12) Patent Application Publication
(10) Pub. No.: US 2005/0215498 A1
(43) Pub. Date: Sep. 29, 2005

(54) Method for the protection of endothelial and epithclial cells during chemotherapy

(76) Inventors: Guenther Eissner, Regensburg (DE); Ernst Holler, Sinzing (DE)

Correspondence Address: MOORE & VAN ALLEN PLLC, P.O. BOX 13706, Research Triangle Park, NC, 27709, US

(21) Appl. No.: 10/516,381

(22) PCT Filed: Jun. 2, 2003
(86) PCT No.: PCT/EP03/05753

§ 371 Date: Jun. 10, 2005

Related U.S. Application Data
(60) Provisional application No. 60/384,114, filed on May 31, 2002.
(60) Provisional application No. 60/387,438, filed on Jun. 11, 2002.

(51) Int. Cl. A61K 48/00; A61K 38/13; A61K 31/7072; A61K 31/7048; A61K 31/704

(52) U.S. Cl. 514/44; 514/11; 514/27; 514/34; 514/283; 514/291; 514/49; 514/109; 514/449

(57) ABSTRACT

The present invention is directed to the use of a protective oligodeoxyribonucleotide for the treatment of a patient undergoing treatment with an immunosuppressant. The invention is further directed to a pharmaceutical composition containing a therapeutically effective dose of an immunosuppressant and of a protective oligodeoxyribonucleotide.

### FIELD OF THE INVENTION

The invention relates to the field use of radiation therapy and/or chemotherapy. More specifically, the invention relates to a method for assuaging side effects associated with such treatment.

### STATE OF THE ART

Allogeneic stem cell transplantation (SCT) is a well established method for the treatment of hematological neoplasias and an increasing variety of other malignant disorders. SCT mainly consists of two sequential steps: The pretransplant conditioning, classically consisting of total body irradiation (TBI) and chemotherapy, leading to minimal residual disease and the immunosuppression of the recipient as the first step, and the transfer of allogeneic stem cells that should finally provide the cure as the second step. However, due to disparities in major (MHC) and minor (mHAg) histocompatibility antigens, severe inflammatory reactions, including acute graft-versus-host disease (GvHD), can occur in different phases post transplant. Based on studies by the inventors¹ and several other investigators^{2,3}it is widely accepted that conditioning contributes via non-specific inflammation to these transplant-related complications (TRC). In addition, a direct toxicity of especially TBI has been demonstrated.^{4,5} This has led to a variety of alternative conditioning regimens currently under investigation. In addition, new pre transplant therapies allow the extension of treatment protocols and the patients' selection. One compound of these novel conditioning concepts is fludarabine, a non-myeloablative immunosuppressant that had originally been used for the treatment of chronic lymphatic leukemia.⁶ Fludarabine in combination with e.g. BCNU and melphalan, cyclophosphamide or other agents can replace TBI or is used together with dose reduced TBI regimens.^{7,8} The clinical data obtained so far argue for comparably low side effects and a hematopoetic and immune cell specificity of fludarabine.⁹ However, the influence of this compound on non-hematopoetic cells like endothelial and epithelial cells has not been subject to investigation yet.

Virtually all TRC are associated with endothelial dysfunction and damage¹⁰. The inventors and others have shown that the endothelium is a target of pre-transplant conditioning in vitro and in vivo. Ionizing radiation induces programmed cell death (apoptosis) in endothelial cells.¹¹⁻¹⁴ At the same time the endothelium is activated in terms of adhesion molecule expression leading to increased leukocyte-endothelial interactions as a prerequisite for inflammatory processes.^{15,16} These effects are significantly enhanced by bacterial endotoxin (lipopolysaccharide, LPS) that might translocate through damaged mucosal barriers from the gastrointestinal tract.¹⁷ In addition, LPS has been shown to increase the antigenicity of endothelial cells towards allogeneic CD8+ cytotoxic T lymphocytes.¹⁸

Clinical results with fludarabine containing reduced intensity conditioning (RIC) regimens obtained so far show a clear downregulation of conditioning-related toxicity without affecting immune reconstitution.²⁵ The incidence of acute GvHD in patients receiving RIC is comparable or even less than in those patients receiving the classical conditioning regimen.²⁶ However, reports on equally severe or even increased late effects like osteonecrosis,²⁷ pulmonal complications,²⁸ and more cases of chronic GvHD²⁹ clearly demonstrate the potential for serious side effects associated with flu-darabine treatment.

### SUMMARY OF THE INVENTION

The invention is based on the discovery that fludarabine activates and damages endothelial and epithelial cells. The activation of the cells leads to damage in the treatment situation where fludarabine is used, e.g., when treating malignancies using SCT. The epithelial and endothelial cells can be protected from this activation and damage by treatment with defibrotide. This treatment may be concomitant or defibrotide may be given before treatment with fludarabine or thereafter.

### ABBREVIATIONS AND DEFINITIONS

SCT: Haematopoetic stem cell transplantation.

Immunosuppressant: substance that down-regulates the immune response of a subject upon administration. Immunosuppressants are used in suppressing the immune system of patients undergoing stem cell therapy. Examples of immunosuppressants include fludarabine, cyclophosphamide, BCNU, cyclosporin, sirolimus, tacrolimus and melphalan. Preferred within the context of this application is fludarabine (also known as 2-fluoro-9-β-D-arabinofuranosyladenine).

Protective oligodeoxyribonucleotide: shall mean, within the context of this application, both oligodeoxyribonucleotides as defined in U.S. Pat. No. 5,646,268 and polydeoxyribonucleotides as defined in U.S. Pat. No. 5,223,609, which are incorporated by reference herein in their entirety.

U.S. Pat. No. 5,646,268 discloses a process for producing an oligodeoxyribonucleotide having the following physico-chemical and chemical characteristics:

| | |
|---|---|
| Molecular weight: | 4000-10000 |
| h: | <10 |
| A + T/C + G* | 1.100-1.455 |
| A + G/C + T* | 0.800-1.160 |
| Specific rotation: | +30°-+48° |

*base molar ratio
h = hyperchromicity parameter

A process for producing such an oligodeoxyribonucleotide comprises: precipitating 0.8M sodium acetate aqueous solutions of polydeoxyribonucleotide sodium salts at 20° C. by addition of an alkyl alcohol selected from the group consisting of ethyl, propyl and isopropyl alcohol.

U.S. Pat. No. 5,223,609 discloses a defibrotide which fulfills certain pharmacological and therapeutical properties and is therefore particularly suitable, if the nucleotide fractions forming it are in stoichiometrical agreement with the following polydeoxyribonucleotidic formula of random sequence:
P₁₋₅, (dAP)₁₂₋₂₄, (dGp)₁₀₋₂₀, (dTp)₁₃₋₂₆, (dCp)₁₀₋₂₀
   wherein
P=phosphoric radical
dAp=deoxyadenylic monomer
dGp=deoxyguanylic monomer
dTp=deoxythymidylic monomer
dCp=deoxycytidylic monomer

The Defibrotide corresponding to this formula moreover shows the following chemico-physical properties: electrophoresis=homogeneous anodic mobility; extinction coefficient, E _{1 cm}^{1%} at 260 ±1 nm=220 ±10; extinction ratio, E₂₃₀/E₂₆₀=0.45±0.04; coefficient of molar extinction (referred to phosphorous), ε(P)=7.750 ±500; rotatory power [α]_{D}^{20°=53 °±6}; reversible hyperchromicity, indicated as % in native DNA, h=15±5.

A preferred protective oligodeoxyribonucleotide is Defibrotide (CAS Registry Number: 83712-60-1), a polynucleotide well known to the person skilled in the art, which normally identifies a polydeoxyribonucleotide obtained by extraction (U.S. Pat. No. 3,770,720 and U.S. Pat. No. 3,899,481) from animal and/or vegetable tissue; this polydeoxyribonucleotide is normally used in the form of a salt of an alkali metal, generally sodium, and usually has a molecular weight of approximately 45-50 kDa. Defibrotide is used principally for its antithrombotic activity (U.S. Pat. No. 3,829,567) although it may be used in different applications, such as, for example, the treatment of acute renal insufficiency (U.S. Pat. No. 4,694,134) and the treatment of acute myocardial ischaemia (U.S. Pat. No. 4,693,995). U.S. Pat. No. 4,985,552 and U.S. Pat. No. 5,223,609 describe a process for the production of defibrotide which enables a product to be obtained which has constant and well defined physico-chemical characteristics and is also free from any undesired side-effects.

### DETAILED DESCRIPTION OF THE INVENTION

The invention relates to a method for the treatment of a patient undergoing treatment with an immunosuppressant, comprising the step of administering an effective dose of a protective oligodeoxyribonucleotide to the patient. The treatment with an immunosuppressant preferably occurs during SCT. The immunosuppressant is preferably selected from the group comprising antimetabolites (e.g., 5-fluorouracil (5-FU), methotrexate (MTX), fludarabine, anti-microtubule agents (e.g., vincristine, vinblastine, taxanes (such as paclitaxel and docetaxel)), alkylating agents (e.g., cyclophasphamide, melphalan, bischloroethylnitrosurea (BCNU)), platinum agents (e.g., cisplatin (also termed cDDP), carboplatin, oxaliplatin, JM-216, CI-973), anthracyclines (e.g., doxorubicin, daunorubicin), antibiotic agents including mitomycin-C, topoisomerase inhibitors (e.g., etoposide, camptothecin), cyclosporin, tacrolimus, sirolimus, and other cytotoxic agents that act to suppress the immune system. A review of such agents that are frequently used in the therapy of malignancies may be found in Gonzales et al., Alergol. Immunol. Clin. 15, 161-181, 2000, which is incorporated herein by reference. Preferred immunosuppressants are nucleosides (i.e. the glycosides resulting from the removal of the phosphate group from a nucleotide), as for instance fludarabine which, by the way, is the preferred immunosuppressant for the purposes of the present invention.

The protective oligodeoxyribonucleotide may be administered concurrently, simultaneously, or together with the immunosuppressant. A preferred combination is the simultaneous gavage of defibrotide and fludarabine.

The step of administering the protective oligodeoxyribonucleotide preferably occurs concurrently, concomitantly, simultaneously, after or before the gavage of the immunosuppressant to the patient.

In a preferred embodiment of the invention, the step of administering the protective oligodeoxyribonucleotide occurs after gavage of the immunosuppressant to the patient. In a further preferred embodiment, the time delay between step of administering the protective and the gavage of the immunosuppressant to the patient is about one hour to about two weeks. The time delay between the step of administering the protective and the gavage of the immunosuppressant to the patient is preferably about two days to about seven days.

In another preferred embodiment of the invention, the step of administering the protective oligodeoxyribonucleotide occurs before gavage of the immunosuppressant to the patient. Preferably, the time difference between step of administering the protective and the gavage of the immunosuppressant to the patient is about one hour to about two weeks. More preferably, the time difference between step of administering the protective and the gavage of the immunosuppressant to the patient is about two hours to about two days.

The preferred protective oligodeoxyribonucleotide is defibrotide, however, other substances as mentioned above as protective oligonucleotides may be used. The following embodiments define preferred doses for defibrotide; however, similar doses may be used when using a protective oligodeoxyribonucleotide which is not defibrotide. The optimal dose for any protective oligodeoxyribonucleotide will be determined by the attending physician. The experiments described below show the protective effects of defibrotide. The effective dose determined in such experiments may be used as a guide for determining an effective dose for treatment. The defibrotide is preferably administered orally or is injected intravenously.

The preferred dose of of defibrotide is chosen so as to reach a blood level of about 100 μg/mL to 0.1 μg/mL. More preferably, the dose of defibrotide is chosen so as to reach a blood level of about 10 μg/mL to about 100 μg/mL. Most preferably, the dose of defibrotide is chosen so as to reach a blood level of about 100 μg/mL.

In a preferred embodiment of the invention, the dose of defibrotide administered is about 100 mg/kg body weight of the patient to about 0.01 mg/kg body weight. Preferably, the dose of defibrotide administered is about 20 mg/kg body weight of the patient to about 0.1 mg/kg body weight. More preferably, the dose of defibrotide administered is about 15 mg/kg body weight of the patient to about 1 mg/kg body weight. More preferably, a daily dosage of about 12 mg to about 14 mg per Kg. of body weight of the patient is administered. Most preferably, the dose of defibrotide administered is about 12 mg/kg body weight of the patient.

Preferably, administration of a protective oligodeoxyribonucleotide according to the invention according to the invention is able to protect endothelial cells and epithelial cells from the effects of the immunosuppressant. The immunosuppressant preferably activates epithelial cells and endothelial cells and induces apoptosis therein. Thus, in a preferred embodiment, the protecting olideoxynucleotide protects epithelial and/or endothelial cells from apoptosis and/or activation by the immunosuppressant. The immunosuppressant is preferably fludarabine. The protective oligodeoxyribonucleotide is preferably defibrotide.

The activation includes enhanced expression of ICAM-1 and of MHC class I molecules. The enhancement of expression is preferably substantial. Further preferably, the immunosuppressant induces a pro-inflammatory activation of endothelial cells and/or of epithelial cells in a patient. The cells are preferably human microvascular endothelial cells (HMEC) and/or dermal and/or alveolar epithelial cells. The damage preferably occurs when the patient's endothelial and/or epithelial cells have been exposed to the immunosuppressant for about 1 hour to about 1 week or more. More preferably, said damage occurs when said cells have been exposed for about 5 hours to about 72 hours. Even more preferably, the duration of such exposure is between 20 hours and 72 hours. Most preferably, the duration of such exposure is more than 48 hours.

The treatment with the immunosupressant preferably occurs during haematopoetic stem cell transplantation. The haematopoetic stem cell transplantation is preferably allogeneic haematopoetic stem cell transplantation.

The invention also relates to a pharmaceutical composition comprising at least a protective oligodeoxynucleotide, for the treatment of a patient in need thereof, which patient is being treated with an immunosuppressant. The administration of said pharmaceutical composition alleviates or protects from side effects caused by the immunosuppressant or by the immunosuppressant and a transplant. The transplant is preferably a bone marrow or haematopoetic stem cell transplant. More preferably, the transplant is an allogeneic bone marrow or haematopoetic stem cell transplant.

The side effects are preferably related to endothial and/or epithelial cells and/or tissues of the patient. Preferably, said side effects involve apoptosis of said cells, and/or activation of said cells. The activation preferably comprises enhanced expression of MHC class I molecules and/or of intercellular adhesion molecule 1 (ICAM-1). The side effects damages human microvascular endothelial cells (HMEC) as well as, preferably, dermal and alveolar epithelial cell lines after 48 hours of culture, when used in pharmacologically relevant concentrations (range: 10 μg/mL to 1 μg/mL).

The side effects generally include damages to target tissues of transplant related complications and stimulated allogeneic immune responses.

The side effects preferably include significant upregulation of the intercellular adhesion molecule 1 (ICAM-1) and MHC class I molecules in endothelial cells and/or peithelial cells of the patient, particularly in alveolar endothelial cells. The side effects further include a a pro-inflammatory activation of microvascular endothelial cells. The side effects further preferably include enhanced lysis of such cells by by allogeneic MHC class I restricted cytotoxic T lymphocytes derived from the transplant.

Adminstration of the protective oligodeoxynucleotide preferably protects against immunosuppressant-induced side effects, including apoptosis and alloactivation.

The pharmaceutical compositions comprising the immunosuppressant of the present invention can be formulated with techniques, excipients and vehicles of conventional and well known type, for the administration both orally and by injection, particularly by intravenous route. The dosages of active ingredient in the compositions according to the present invention ranges between 50 and 1500 mg for unitary dose, whereas to attain the desired results the daily administration of 10 to 40 mg/kg is suggested. Methods for the preparation defibrotide may be found in U.S. Pat. No. 4,985,552 and U.S. Pat. No. 5,223,609, which patents are incorporated hereby in their entirety by reference.

The invention also relates to a pharmaceutical composition containing a therapeutically effective dose of an immunosuppressant and of a protective oligodeoxyribonucleotide. The immunosuppressant is preferably fludarabine. protective oligodeoxyribonucleotide is preferably defibrotide.

### BRIEF DESCRIPTION OF THE FIGURES

FIG. 1: Fludarabine induces programmed cell death in human micorvascular endothelial cells (HMEC). HMEC were either left untreated or incubated with 2-fluoro-9-β-D-arabinofuranosyladenine (hereinafter referred to as F-Ara, the metabolized form of fludarabine) in descending concentrations for 48 hours and subjected to flow cytometric analysis (A) or microscopic DAPI stain analysis. A: Contour plots of the side scatter (SSC) image (x-axis) of propidium iodide (PI)-negative cells plotted against the forward scatter image (y-axis) as a parameter for cellular granularity versus cell size. B: Quantitative fluorescence microscopy analysis of DAPI-stained endothelial cells. Results are given in % apoptotic HMEC (% apoptotic cells) ± standard deviation (out of n=10 microscopic fields with an average of 70 cells per field). Representatives of at least five independent experiments are shown. *: p<0.001 of untreated control versus F-Ara (10 μg/mL) treated cells.

FIG. 2: Defibrotide (D) inhibits F-Ara-induced apoptosis in HMEC, evidence for an intracellular antagonism. F-Ara: 10 μg/mL. D: 100 μg/mL. Flow cytometric analysis of the SSC-image of PI-negative cells. A: reproducible induction of apoptosis by F-Ara. B: Dose-dependent inhibition of F-Ara-induced apoptosis by D. C: left plot: incubation of HMEC with F-Ara for 1 hour, subsequent incubation with D for 48 hours after washing. Right plot: incubation of HMEC with D for 1 hour, subsequent incubation with F-Ara for 48 hours after washing. For experimental details see legend to FIG. 1 and Materials and Methods. Shown is one representative out of three independent experiments.

FIG. 3: F-Ara induces apoptosis in keratinocytes and alveolar epithelial cells, but not in gut or bronchial epithelial cells; protective effect of Defibrotide. F-Ara: 10 μg/mL. D: 100 μg/mL. Flow cytometric analysis of the SSC-image of PI-negative cells (FIG. 3 A) and DAPI-stain analysis of apoptotic cells (FIG. 3 B). Results are given in mean % apoptotic cells ± standard deviation out of three different experiments. HaCaT: human keratinocyte cell line. SW 480: gut epithelial cells line. A 549: lung carcinoma cell line from the alveolar epithelium. BEAS-2B: bronchial epithelial cell line. Primary bronchial epithelial cells have been derived from a bronchoscopic brush procedure. FIG. 3 A: *: p=0.005 of F-Ara- versus F-Ara+D treated HaCaT cells. **: p=0.1 16 of F-Ara versus F-Ara+D treated A 549 cells. [symbol] H: no apoptosis induction. FIG. 3 B: +: p=0.026 of F-Ara- versus F-Ara+D treated HaCaT cells. ++: p=0.001 of F-Ara versus F-Ara+D treated A 549 cells. For experimental details see legend to FIG. 1 and Materials and Methods. Three representative experiments are summarized for each cell line.

FIG. 4: Defibrotide (D) Does Not Interfere with the Anti-Leukaemic and the Anti-PBMC Effect of F-Ara. F-Ara: 10 μg/mL. D: 100 μg/mL. A: Propidium iodide staining of primary acute myeloid leukemia (AML) cells derived from a patient in blast crisis (70% blasts of total PBMC count). Results are given in mean % vitality of three independent experiments. *: p=0.008 of Control- versus F-Ara-treated AML cells. B: Flow cytometric analysis of the SSC-image of PI-negative PBMC. Shown is one representative out of five independent experiments with different blood donors.

FIG. 5: F-Ara Induces ICAM-1 Expression on HMEC, Protective Effect of Defibrotide (D). Flow cytometric analysis of ICAM-1 positive cells. HMEC were either left untreated or incubated with F-Ara (10 μg/mL, or descending concentrations in B) in the presence or absence of descending concentrations of D. A: Histogram plot of ICAM-1 expression from a representative experiment. Dotted line: Background staining (nil control); thin line: ICAM-1 expression of untreated control cells; thick line: ICAM-1 expression of F-Ara-treated cells. B: Dose-dependent induction of ICAM-1 expression by F-Ara. Summary of three independent experiments. Results are given as mean % ICAM-1 positive cells ± standard deviation. *: p=0.075 of F-Ara- versus untreated control cells. C: Dose-dependent inhibition of F-Ara-induced ICAM-1 expression by Defibrotide (D). Results are given as mean % ICAM-1 positive cells ± standard deviation. **: p=0.004 of F-Ara- versus F-Ara+D-treated HMEC.

FIG. 6: F-Ara Increases the Allogenicity of HMEC for CD8-positive cytotoxic T-lymphocytes (CTL), Protective Effect of Defibrotide. A: PBMC were stimulated with irradiated HMEC in the presence of interleukin 2 (50 U/mL) for 7 days and subjected to a ⁵¹Cr release assay with untreated (Control) and F-Ara (10 μg/mL)-treated HMEC (24 hour-incubation) as target cells. autolog. B-LCL: autologous (effector) EBV-transformed B-lymphoblastoid cells. K 562: target cells for natural killer (NK) cells. Results are given as % specific lysis as described in Materials and Methods. *_: % specific lysis of F-Ara-treated HMEC in the presence of anti-MHC class I antibody w6/32. E/T ratio: effector/target ratio. B: Downregulation of F-Ara-induced allogenicity of HMEC towards CD8-positive CTL by Defibrotide (D). CD8-positive PBMC have been negatively selected (non-CD8+-cell-depleted) by magnetic bead separation. For experimental details see legend to FIG. 6 A.

FIG. 7: F-Ara decreases the allogenicity of HMEC for NK Cells, enhancement of lysis by blockade of MHC class I. NK cells have been negatively selected (non-NK-cell-depleted) by magnetic bead separation and stimulated with irradiated HMEC in the presence of IL-2 (50 U/mL) for 4 days and subsequently subjected to a ⁵¹Cr release assay as described for FIG. 6. Table below the graph: Flow cytometric analysis of the effector cell population pre and post stimulation with HMEC. NK cells were characterized as CD3−/CD16+CD56+. *_: % specific lysis of K 562 cells at E/T ratio of 20:1.

TABLE 1
Anti-endothelial CTL elicit a T_{c}1-like phenotype.

| Effector | IFN-γ | IL-4 |
|---|---|---|
| PBMC | 319 [±176] | 0 |
| CD8+ | 524 [±174] | 0 |

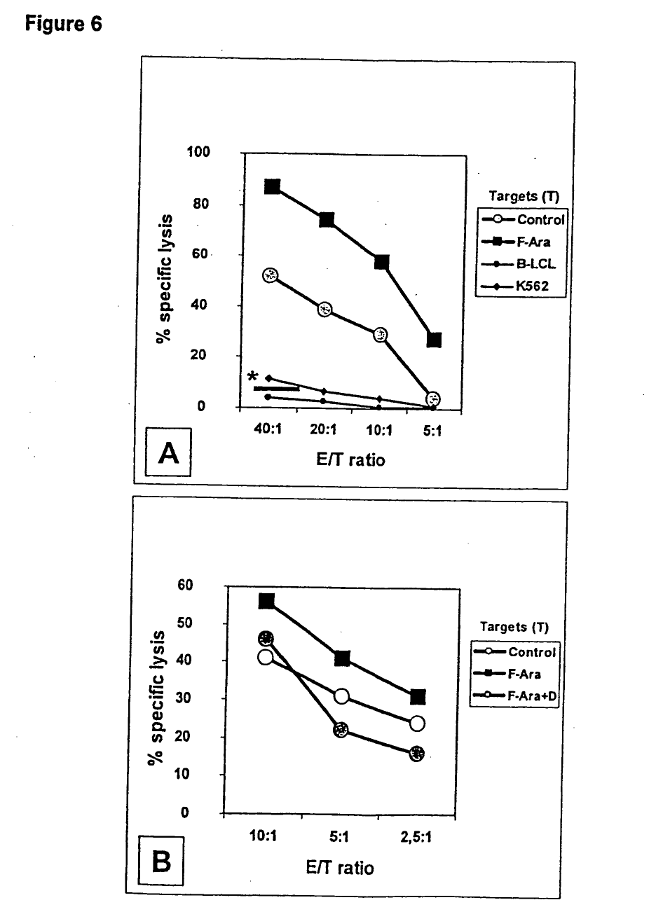

ELISA for the production of interferon gamma (IFN-γ) and interleukin 4 (IL-4) in the supernatants of stimulated effector cells (7 days, irradiated HMEC, 50 U/mL IL-2). PBMC were either left unseparated or negatively selected for CD8+ T cells as given for the experiments in FIG. 6. Results are given as mean pg/mL cytokine ± standard deviation of 3 independent experiments.

### EXAMPLES

Methods

Cell Culture and Reagents

The human dermal microvascular endothelial cell line CDC/EU.-HMEC-1 (further referred to as HMEC) was kindly provided by the centres for Disease Control and Prevention (Atlanta, Ga., USA) and has been established as previously described.¹⁹ HMEC were cultured in MCDB131 medium, supplemented with 15% fetal calf serum (FCS), 1 μg/mL hydrocortisone (Sigma, Deisenhofen, Germany), 10 ng/mL epidermal growth factor (Collaborative Biochemical Products, Bedford, Mass., USA) and antibiotics. All cell culture reagents have been purchased by Gibco BRL (Karlsruhe, Germany) unless stated otherwise. 2-Fluoroadenine 9-beta-D-arabinofuranoside (F-Ara) was obtained from Sigma, Deisenhofen, Germany, Defibrotide vials were obtained from Prociclide™, Crinos, Como, Italy.

Apoptosis Assays

An established method for detecting apoptosis in human endothelial cells was performed as previously described²⁰ using flow cytometry (FACScan™ and CellQuest™ software, Becton Dickinson, Heidelberg, Germany). Endothelial and epithelial cells were either left untreated or incubated with F-Ara in descending concentrations (range: 10 μg/mL to 0.1 μg/mL) in the presence or absence of Defibrotide for 48 hours. Afterwards, cells were washed in PBS/10% FCS and stained with the necrosis detecting dye propidium iodide (PI, 0.2 μg/mL, Sigma, Deisenhofen, Germany). Apoptotic cells were identified by a PI-negative staining and by a characteristic side scatter image distinct from that of non-apoptotic cells. At least three experiments per cell type have been performed.

An alternative method for the detection of apoptosis used microscopic analysis of DNA fluorescence labeled cells. 1×10 ⁵/plate endothelial cells were seeded in 35 mm petri dishes (Nunc, Wiesbaden, Germany). These cells were treated as given above and subsequently fixed with Methanol/Acetone (1:1) for 2 minutes, washed once in PBS and stained with 4,6-Diamidino-2-phenylindole (DAPI) (0.5 μg/mL, Sigma, Deisenhofen, Germany), dissolved in 20% Glycerin/PBS. Samples were mounted and subjected to microscopic analysis. Nuclear condensation as revealed by DAPI staining in the absence of trypan blue uptake is considered characteristic of apoptosis as opposed to necrosis.²¹ The quantitative analysis included counting the number of apoptotic relative to all identifiable cells from at least 10 microscopic fields, with an average of 70 cells per field.

For the sake of the clarity of the manuscript DAPI stain results are only displayed for the experiments with endothelial cells and HaCaT as well as A 549 cells.

### CELL SURFACE ANALYSES

Cell surface expression of ICAM-1 (Becton Dickinson/Pharmingen, Heidelberg, Germany) and MHC class I (w6/32, hybridoma supernatant, ATCC, Manassas, Va., USA) molecules on HMEC was assessed by the indirect immunofluorescence technique and subsequent flow cytometry using the FACScan™ flow cytometer and the Cell-Quest™ analysis program (Becton Dickinson, Heidelberg, Germany). Endothelial cells were treated as given and after incubation harvested with trypsin/EDTA (Gibco), washed once in cold PBS/10% FCS and incubated 1 hour on ice with 5 μg/mL of anti-adhesion molecule MoAbs. Cells were washed again and incubated with a goat anti-mouse IgG-FITC conjugated antibody F(ab)₂ fragment (Dako, Hamburg, Germany) for 45 minutes on ice. Cells were then washed in PBS/10% FCS and subjected to analysis. Viability of the cells was determined by concurrent propidium iodide (0.2 μg/mL, Sigma, Deisenhofen, Germany) staining. Omitting of the first antibody served as a negative control to detect unspecific fluorescence. This approach, instead of using isotype control antibodies, was justified by previous observations that endothelial cells lack Fc receptors.22 Therefore, a non-specific binding of antibodies through their Fc portion could be excluded.

Allostimulation of Peripheral Blood Cells with Hmec

Peripheral blood mononuclear cells (PBMC) were derived from heparinized (Novo Nordisk, Mainz, Germany) blood of healthy human volunteers or buffy coats from the Bavarian Red Cross according to a standard protocol using Ficoll hypaque (Pharmacia, Freiburg, Germany) density gradient centrifugation. Cells were then stimulated in a ratio of 1:1 and 2:1 with irradiated (20 Gy) HMEC for 7 days in the presence of Interleukin 2 (50 U/mL) and 10% human AB serum (Sigma, Deisenhofen, Germany). Alternatively, PBMC were selected for CD8+ T cells and natural killer (NK) cells using cell isolation kits according to the manufactuer's instructions (MACS™, Miltenyi Bio-tech, Bergisch-Gladbach, Germany) based on the deletion of non CD8+ and non NK cells, respectively. Stimulation of the selected cells was identical to that of whole PBMC cultures, except for NK cells which were stimulated for only 3 days.

Cytotoxicity Assay

T cell- or NK-cell mediated cytotoxicity was assessed according to a well established protocol,²³ using a 4h ⁵¹Cr radioisotope assay. HMEC that had either been left untreated or incubated with F-Ara (10μg/mL) overnight were used as target cells, to be labeled 0.4 mCi Na₂⁵¹CrO₄ for 2 hours. After 3 washing steps, target cells were adjusted to 10⁴ cells/mL and coincubated with PBMC, CD8+ or NK effector cells at descending effector to target ratios for another 4 hours. Supernatants were transfered to dry scintillation plates and counted in a γ-counter (all from Canberra Packard, Darmstadt, Germany). Autologous (effector) B-Lymphoblastoid cell lines (B-LCL) and K562 as NK sensitive cells were taken as additional control targets. The percentage of specific lysis was calculated as: [(experimental release−spontaneous release)/(maximal release−spontaneous release)]×100. Spontaneous release in all experiments was always below 20%.

Enzyme Linked Immunosorbent Assays (Elisa)

The ELISA for the detection of Interleukin 4 (IL-4, T_{c}2 response) and Interferon γ (IFNγ, T_{c}1 response), IL-1 and IL-1 0 in the supernatants of allogeneic effector T cells (see below) were performed exactly according to the manufacturer's kit instructions (R&D Systems, Minneapolis, Minn., USA).

Statistical Analysis

The significance of differences between experimental values was assessed by means of the Student's t-test.

### Example 1

### F-Ara Induces Apoptosis (Programmed Cell Death) in Human Microvascular Endothelial Cells (HMEC)

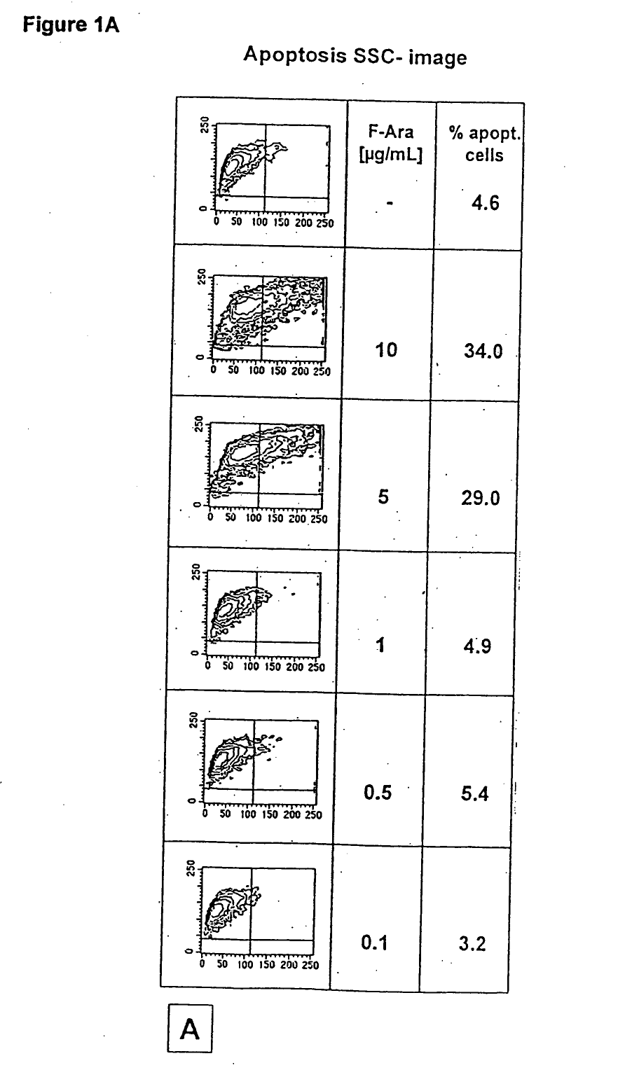

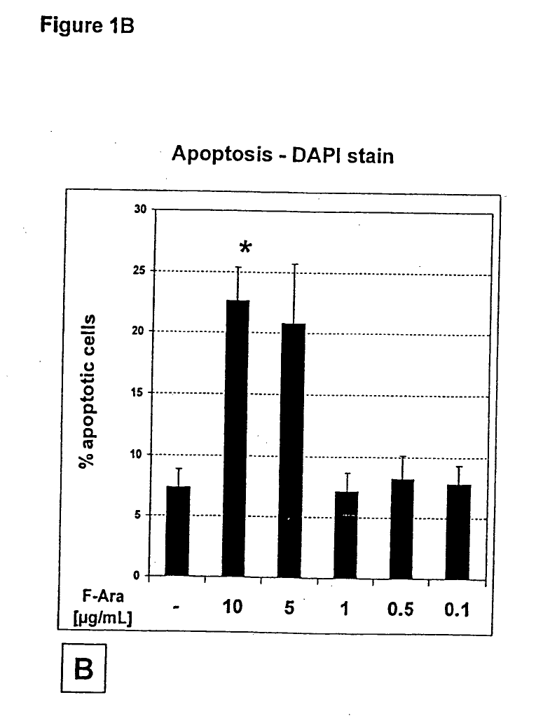

In order to assess the influence of F-Ara on the viability of cultured human endothelial cells, HMEC were incubated with descending pharmacologically relevant concentrations (10 μg/mL to 0.1 μg/mL) of 2-Fluoroadenine 9-beta-D-arabinofuranoside as the metabolized form of fludarabine. The median intracellular level of the active (cytotoxic) fludarabine triphosphate in target cells is 20 μM, representing a concentration 5.8 μg/mL (medac SCHERING, manufacturers's instructions). After 48 hours of incubation HMEC were subjected to apoptosis assays using the detection of cellular granularity of propidium iodide negative cells (side scatter (SSC) image in flow cytometry) and microscopic analyses of DAPI-stained cells, respectively. Independent of the assays system, FIG. 1 A and B clearly demonstrate that F-Ara causes apoptosis in HMEC in concentrations of 10 and 5 μg/mL, whereas 1 μg/mL was no longer effective. The critical threshold of the cytotoxicity of F-Ara was between 2 and 3 μg/mL. Apoptosis by F-Ara was already detectable after 24h, though to a lesser extent (data not shown).

### Example 2

### Defibrotide Protects HMEC from the F-Ara Induced Apoptosis

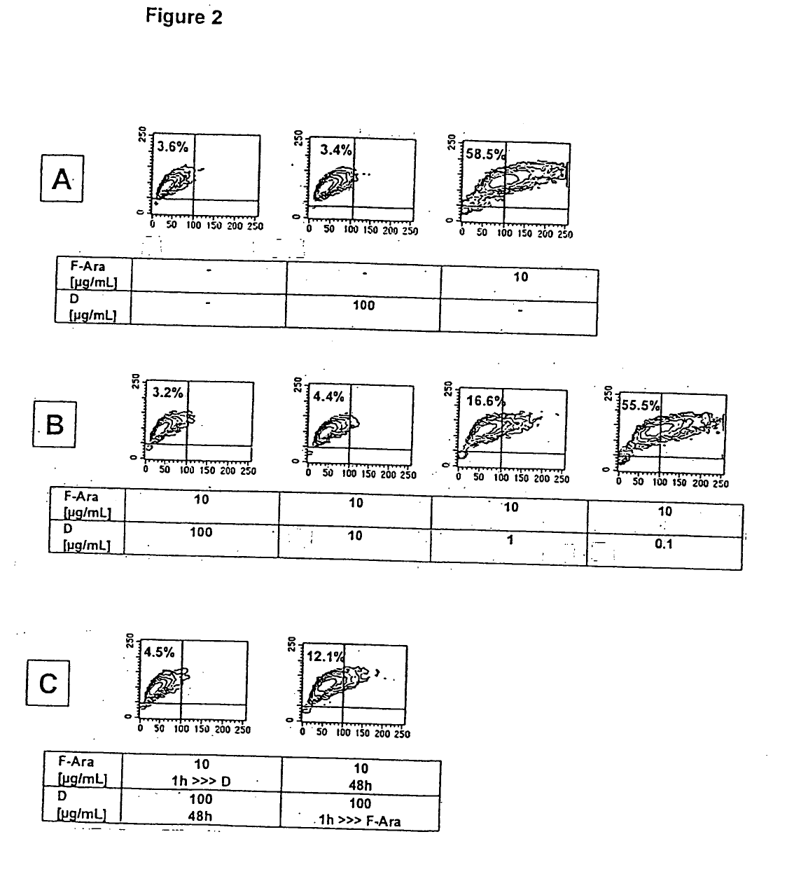

HMEC had either been left untreated or treated with F-Ara in the presence or absence of varying concentrations of Defibrotide (100 μg/mL to 0.1 μg/mL) and assessed for programmed cell death after 48 hours using flow cytometric analyses of the SSC image as described for FIG. 1 A. FIG. 2 A (mid contour plot) shows that Defibrotide alone as a second control did not influence endothelial cell viability. The apoptotic effect of F-Ara is reproduced in FIG. 2 A (right contour plot), whereas FIG. 2 B shows a dose-dependent protection of F-Ara induced cell death by Defibrotide. In order to exclude unspecific artifical extracellular interaction of F-Ara and Defibrotide in vitro HMEC were pretreated with Defibrotide for 1 hour and subsequently, after 3 washing steps, incubated with F-Ara for another 48 hours and vice versa. FIG. 2 C (right contour plot) reveales that pretreatment of HMEC for 1 hour was sufficient to protect cells from F-Ara induced apoptosis. Similarly, pretreatment of HMEC with F-Ara for 1 hour (FIG. 2 C, left contour plot) and subsequent incubation with Defibrotide did not lead to endothelial programmed cell death.

### Example 3

### Effect of F-Ara on Different Epithelial Cell Lines, Protective Effect of Defibrotide

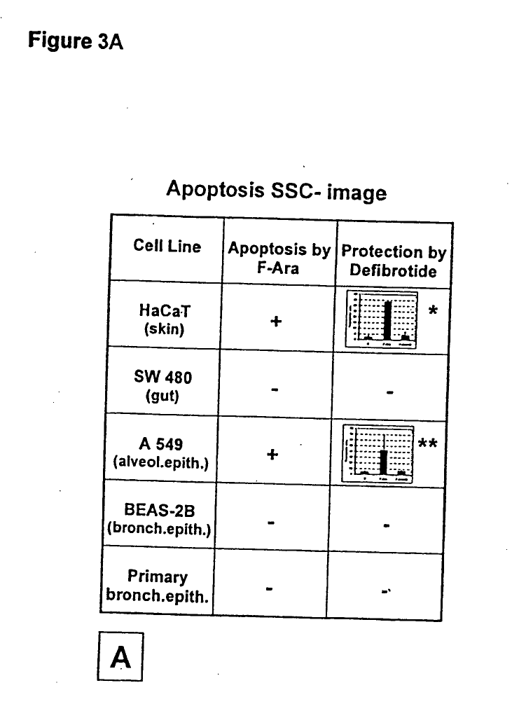

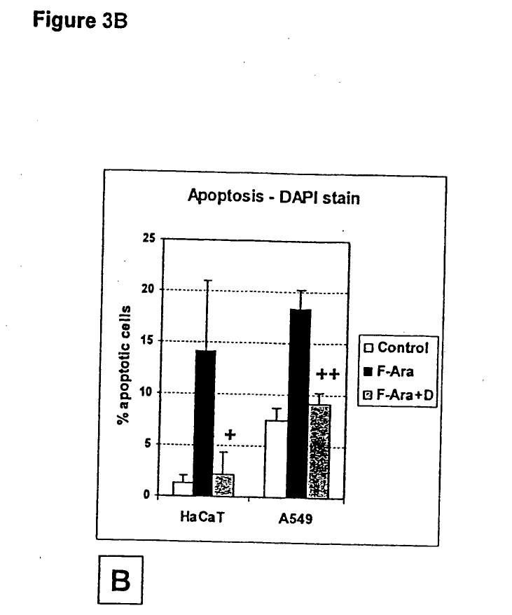

Skin, the gastrointestinal tract (GIT) and most likely the lung are among the primary targets of GvHD. Therefore, it was reasonable to test the influence of F-Ara on cell lines derived from these organs. Cells from keratinocyte (HaCaT), GIT (SW 480), alveolar (A549) and bronchial epithelial (BEAS-2B) cell lines as well as primary bronchial epithelial cells were incubated with F-Ara (10 μg/mL) as given for FIGS. 1 and 2 and assayed in flow cytometric apoptosis analyses 48 hours post treatment. FIG. 3 A summarizes that gut and bronchial epithelial cells appeared to be resistant to the apoptotic stimuli of F-Ara, whereas keratinocytes (HaCaT) and alveolar epithelial cells (A549) showed signs of apoptosis, as determined by flow cytometry of the SSC image (34.0 [±1.0] % apoptotic cells for HaCaT and 42.9 [±26.7] % for A549, respectively). Again, the protective potential of Defibrotide (100 μg/mL) was assessed. HaCaT (4.3 [±3.0] %) and A 549 (5.4 [±2.9] %) cells were completely protected from programmed cell death after cotreatment with F-Ara and Defibrotide FIG. 3 A, inserted bar graphs). To confirm these results, DAPI-stain apoptosis assays were performed for HaCAT (FIG. 3 B, left columns) and A 549 cells (FIG. 3 B, right columns). As shown for endothelial cells, Defibrotide alone did not influence the number of apoptotic cells in either cell line (data not shown).

### Example 4

### Defibrotide Does Not Interfere with the Anti-Leukaemic and Anti-PBMC Effect of F-Ara

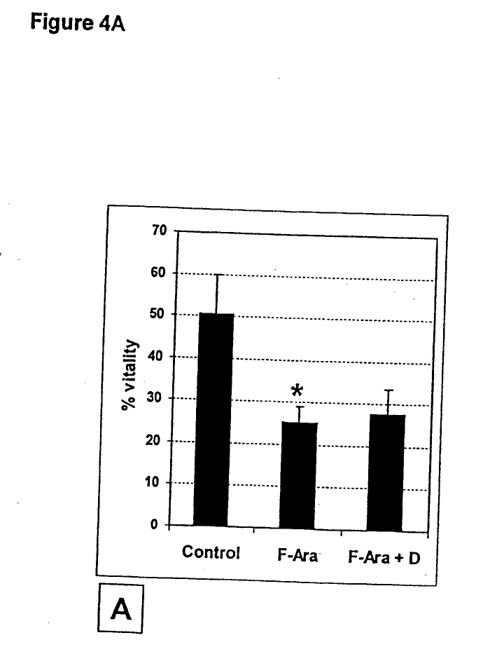

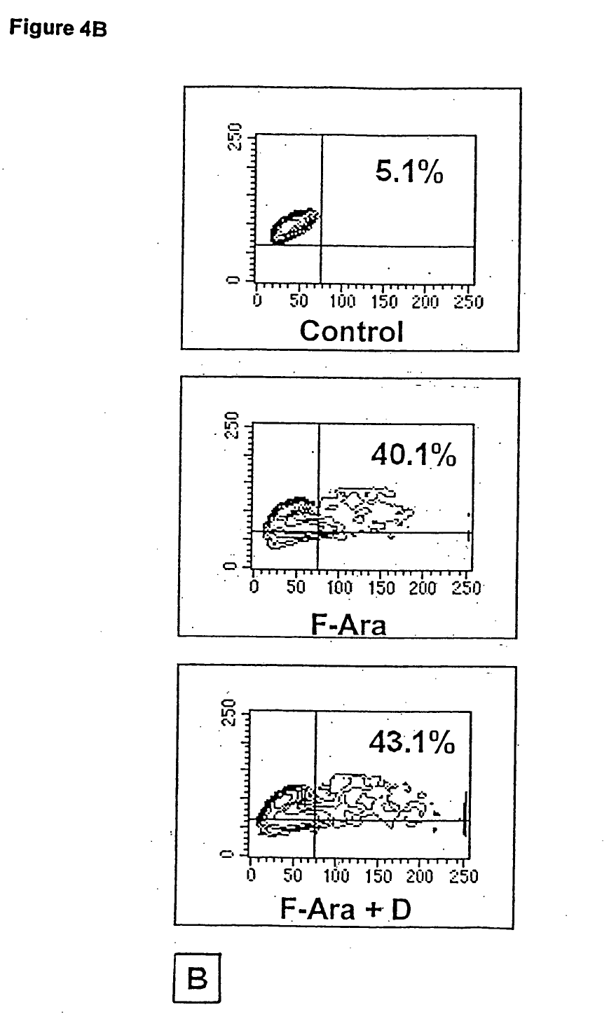

Next to its desirable protective capacity for endothelial and epithelial cells against F-Ara induced apoptosis it was important to investigate whether Defibrotide would also interfere with the anti-leukaemic properties of F-Ara. To address this question, primary peripheral blood derived acute myeloid leukaemic (AML) cells with a blast amount of 70% were thawed, kept in culture for 24 hours and subsequently treated with F-Ara in the presence or absence of Defibrotide for another 48 hours. FIG. 4 A demonstrates that already almost 50% of the cells died spontaneously of necrotic cell death. However, F-Ara induced cell death in up to 80% of the cells. In contrast to its effect on endothelial and epithelial cells, Defibrotide was not able to protect the AML cells from the F-Ara mediated toxicity. It is of note that FIG. 4 A describes % vitality of the cells, not % apoptotic cells, due to the fact that F-Ara directly caused necrosis, rather than apoptosis in AML cells. This could be observed after as early as 24 hours of incubation. Still, FIG. 4 A clearly shows that Defibrotide does not interfere with the desirable toxicity of F-Ara against leukaemic cells. We next asked whether Defibrotide might modulate the effect of F-Ara against normal haematopoetic cells and performed apoptosis assays (SSC-image) with PBMC from normal human blood donors. As could be learned from a representative experiment depicted in FIG. 4 B, F-Ara induced apoptosis in 40.1% of the cells as compared to 5.1% apoptotic cells in the untreated control. Again, Defibrotide did not interfere with the apoptotic activity of F-Ara against PBMC (43.1% apoptotic cells), suggesting that the immunosuppresssive properties of F-Ara are not harmed by cotreatment with Defibrotide.

### Example 5

### F-Ara Upregulates Intercellular Adhesion Molecule 1 (ICAM-1) on HMEC with Antagonistic Effects of Defibrotide

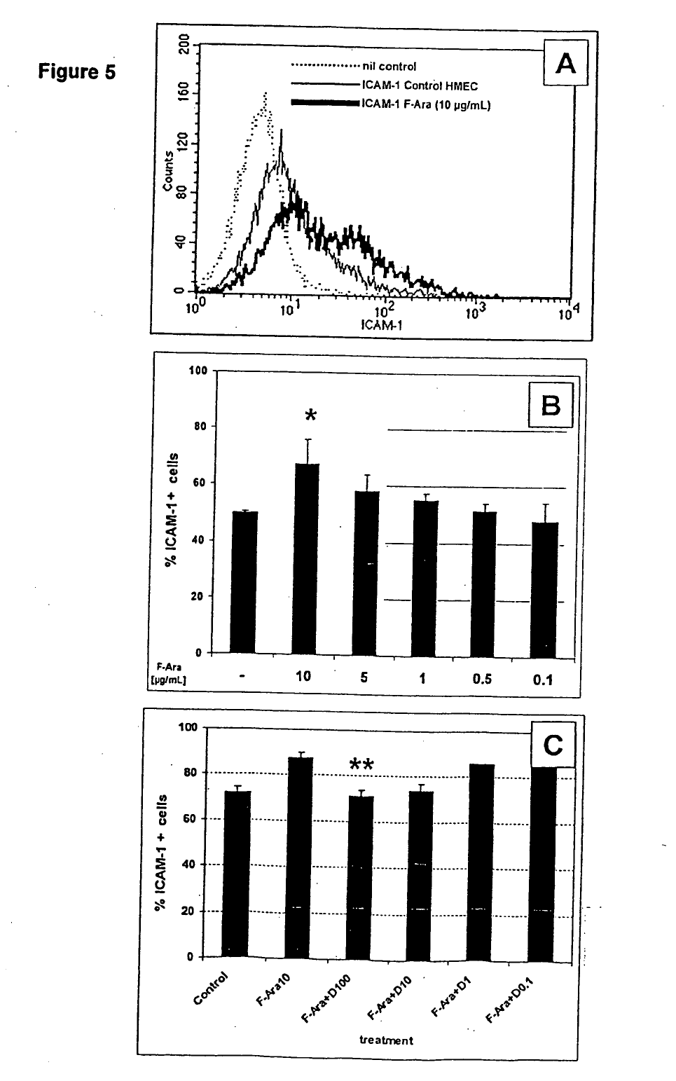

Based on previous observations that pretransplant conditioning not only damages, but also leads to proinflammatory activation of endothelial cells in terms of adhesion molecule induction,¹⁵ we next investigated the expression of ICAM-1 under the influence of F-Ara. As depicted in FIG. 5 A and B, flow cytometric analyses demonstrated that F-Ara, after 24 hours of incubation, significantly enhances expression on HMEC in a dose-dependent manner similar to that observed for apoptosis induction. Concentrations down to 1 μg/mL of F-Ara were effective in inducing ICAM-1. We next asked whether Defibrotide would also be functional as an antagonist of F-Ara in this experimental setting. HMEC were treated with F-Ara as given and incubated in the presence or absence of descending concentrations of Defibrotide. FIG. 5 C summarizes 3 independent experiments showing that Defibrotide in fact antagonized the F-Ara induced ICAM-1 expression in concentrations of 100 μg/mL and 10 μg/mL. It is of note that Defibrotide alone did not activate endothelial cells, the ICAM-1 expresssion remained unchanged with every concentration tested (data not shown).

Since a proinflammatory activation of target cells is often associated with increased expression of major histocompatibility antigens (MHC) class I and II, we did further flow cytometric analyses for these antigens after incubation with F-Ara in various concentrations for 24 hours. Despite its well described immunosuppressive properties, F-Ara surprisingly induced MHC class I molecules on HMEC dose-dependently (1.5 fold induction of mean fluorescence intensity at 10 μg/mL, 1.3 fold induction at 5 μg/mL), whereas MHC class II remained unchanged (data not shown).

### Example 6

### F-Ara Increases the Antigenicity of Endothelial Cells Towards Allogeneic Peripheral Blood Cells, Protection by Defibrotide

The induction of MHC class I molecules on HMEC by F-Ara prompted us to examine whether F-Ara would also enhance the capacity of HMEC to stimulate allocytotoxic responses. Peripheral blood mononuclear cells (PBMC) as effectors were either derived from heparinized blood of healthy human volunteers of from buffy coat preparations, stimulated with irradiated (20 Gy) HMEC in the presence of 50 U/mL interleukin 2 (IL-2) for 7 days and subsequently subjected to a standard ⁵¹Cr release assay (for details see Materials and Methods). At day—1 fresh HMEC as targets were either left unstimulated or incubated with F-Ara (1 μg/mL) in the presence or absence of an anti-MHC class I neutralizing antibody (w6/32). Autologous effector Epstein-Barr virus transformed B-lymphoblastoid cell lines (B-LCL) and K562 cells as classical natural killer (NK) cell targets served as controls. FIG. 6 A demonstrates that F-Ara indeed increased the antigenicity of HMEC towards allogeneic PBMC at all E/T ratios tested. The lack of specific lysis of K 562 and autologous effector B-LCL verified the involvement of MHC restricted cytotoxic T lymphocytes (CTL). In addition, lysis of either untreated or F-Ara treated HMEC could almost fully be blocked after coincubation of these cells with the anti-MHC class I antibody w6/32 (FIG. 6 A, *_). To further confirm that CD8+ CTL were responsible for the anti-endothelial cytotoxic activity, PBMC were selected for CD8+ and CD4+ T cells (non-CD8 and non-CD4-depleted PBMC, respectively) using magnetic bead separation with MACS™ bead kits. Purity of the preparations was≧93% in all cases with a complete absence of the other cell population (not shown). Separated T cells were stimulated with HMEC and IL-2 exactly as described for unselected PBMC (see above). As shown in FIG. 6 B, lysis of F-Ara-treated HMEC by CD8+ CTL was , again, significantly higher than that of control HMEC. Furthermore, pretreatment of target HMEC with F-Ara and Defibrotide (F-Ara+D) downregulated specific lysis even below control levels, suggesting that Defibrotide also protects endothelial cells against the lysis of allogeneic effector lymphocytes. HMEC stimulated CD4+ T cells did not show any signs of cytotoxic activity in this experimental setting (data not shown). Flow cytometric analyses of F-Ara versus F-Ara+D treated HMEC resulted in a significant downregulation of MHC class I molecules by Defibrotide, suggesting that MHC class I expression is the critical element in regulating the cytotoxic response induced by F-Ara (data not shown).

### Example 7

### Anti-Endothelial CTL Display an T_{c}1-Like Phenotype

To gain information about the nature of the anti-endothelial CTL, PBMC and CD8+ T cells were stimulated as given above, and supernatants were collected for the assessment of interferon gamma (IFN-γ) and interleukin 4 (IL-4) using ELISA analyses. As depicted in Tab 1, stimulation with HMEC and IL-2 obviously led to the outgrowth of T_{c}1-like T cells as could be told from the unique expression of IFN-γ, whereas no IL-4 was produced.

### Example 8

### F-Ara Downregulates Lysis of HMEC by Allogeneic NK Cells

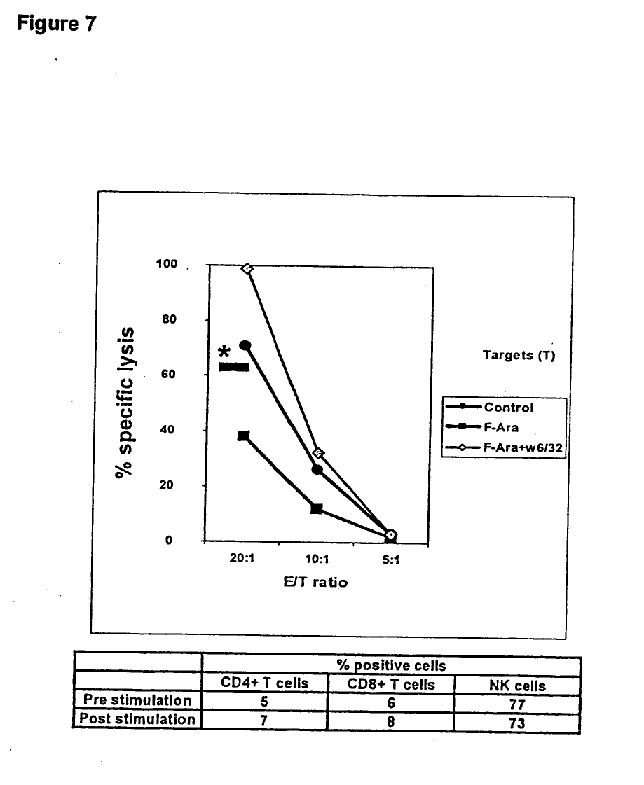

Another interesting question was how F-Ara induced modulations of the MHC class I expression affects the cytolytic response of natural killer (NK) cells against endothelial cells. PBMC from healthy individuals were negatively selected for NK cells (non-NK cell depleted) and stimulated for 4 days with irradiated HMEC in the presence of IL-2, as it was described for the experiment in FIG. 6B. At day 4, HMEC as target cells have either been left untreated or incubated with F-Ara (10 μg/mL) for 24 hours and subjected to a standard ⁵¹Cr release assay with the stimulated NK cells as effectors. FIG. 7 demonstrates that F-Ara significantly downregulated the allogenicity of HMEC towards NK cells. As a positive control for NK cell activity, lysis of MHC class I negative K 562 cells could be observed (FIG. 7, *_). Pretreatment of F-Ara stimulated HMEC with the anti-MHC class I antibody w6/32 completely abrogated the effect of F-Ara and led to almost 100% specific lysis of HMEC (FIG. 7), suggesting that MHC class I on the surface of HMEC is, again, the critical switch for the regulation of the cytotoxic response of NK cells. The role of killer cell inhibitory receptors (KIR) that have been found to be negatively regulated by high expression levels of MHC class I molecules²⁴ might be responsible for the the decreased cytolytic response of NK cells.

Discussion

Clinical results with fludarabine containing reduced intensity conditioning (RIC) regimens obtained so far show a clear downregulation of conditioning-related toxicity without affecting immune reconstitution.²⁵ The incidence of acute GvHD in patients receiving RIC is comparable or even less than in those patients receiving the classical conditioning regimen.²⁶ However, reports on equally severe or even increased late effects like osteonecrosis,²⁷ pulmonal complications,²⁸ and more cases of chronic GvHD arise.²⁹ Despite its well documented immunosuppressive properties fludarabine, in our study, has turned out to activate and damage endothelial and epithelial cells. This observation might, at least in part, explain the undesired clinical side effects described above, since osteonecrosis is an expression of endothelial dysfunction, and fludarabine appears to be toxic for alveolar epithelial cells. It is interesting to note that the harmful effects of fludarabine on lung cells seem to be compartment-specific, as bronchial epithelial cells did not undergo apoptosis in response to this immunosuppressant. The fact that a keratinocyte cell line (HaCaT) was also sensitive to fludarabine suggest that it might also be involved in cutaneous disorders post SCT. As the pathogenesis of late complications is multifactorial and might also be influenced by increasing age of the SCT patients and the use of peripheral stem cells further evaluation in clinical analyses of pulmonal and dermatological complications is needed.

Since in many pre-transplant protocols fludarabine is used in combination with ionizing radiation it was important to test whether these two compounds would cooperate in affecting endothelial cells. Interestingly, we could not find any enhancement of radiation induced cell death by fludarabine or vice versa (data not shown). This suggests differential mechanisms of how the apoptotic death signal is transfered to endothelial cells.

The precise mechanism how fludarabine induces apoptosis in endothelial and epithelial cells remains to be elucidated. It is likely that fludarabine—as a purin analogue—integrates into the DNA and thus causes mutations that lead to gene deletion like reported previously.³⁰ It has also been suggested that fludarabine can cooperate with cytochrome c and apoptosis protein-activating factor-1 (APAF-1) in triggering the apoptotic caspase pathway.³¹

Fludarabine increases the allogenicity of endothelial cell targets for CD8+ T cells. In contrast, Fludarabine significantly downmodulates the endothelial lysis by allogeneic NK cells. The MHC class I expression seems to be critical for the regulation of any of these immune responses, since a blockade of class I fully abrogated CTL lysis and tremendously upregulated lysis by NK cells. These opposing effects of fludarabine, taken together with the clinical observation that fludarabine shows less acute and equal or even more chronic toxicity than the classical conditioning regimen raises the speculation that NK cells and CTL might be active in different phases of GvHD pathophysiology, i.e. NK cells would primarily act in the earlier (suppressed by fludarabine), and CTL in the later phase (enhanced by fludarabine) post transplant.

With regard to the nature of the anti-endothelial CTL it is an interesting question whether these CTL are endothelial- or simply allo-specific. The existence of endothelial-specific effector lymphocytes has been described previously.³² In contrast to the CTL we characterized as displaying a Tc1-like phenotype, many of the CTL clones reported show little, if any, IFN-γ and unusually express CD40 ligand at rest what might enhance cytolytic activity.³³ But these data do not rule out the existence of additional allogeneic CTL with a specificity for non-hematopoetic targets.

Defibrotide is a well tolerated drug successfully used for the treatment of veno-occlusive disease as one major hepatic complication post SCT.34 In addition, there is an increasing number of pre-clinical and clinical reports showing its efficacy in treating ischemia/reperfusion injury and atherosclerosis, as well as recurrent thrombotic thrombocytopenic purpura.³⁵⁻³⁷ Defibrotide is known to act directly on endothelial cells without further metabolism required³⁸ and could, therefore, be used in our in vitro studies. Defibrotide fully protected endothelial and epithelial cells from fludarabine mediated apoptosis. Additional experimentation is needed to assess the precise mechanism of protection by which Defibrotide antagonizes fludarabine, but one can imagine a role for Defibrotide in an inhibition of DNA integration of fludarabine or the aforementioned caspase activation. Besides its anti-apoptotic effects, Defibrotide was able to downregulate anti-endothelial CTL responses by regulating MHC class I expression. In contrast, Defibrotide did not affect the desirable anti-leukemic effect of fludarabine, as shown by the lack of protection of AML cells. Another important observation was that Defibrotide could not block the fludarabine-mediated apoptosis of PBMC. This suggests that the immunosuppressant effect of fludarabine mandatory for conditioning is not influenced by a co-treatment with defibrotide.

It is of note that Defibrotide was not protective against radiation induced endothelial cell damage, suggesting its effect to be specific for fludarabine mediated cellular changes (data not shown).

Based on these results and with respect to its little, if any, side effects,³⁹ we conclude from our study that Defibrotide is a good candidate used in combination with fludarabine during conditioning prior to SCT, especially in patients at risk for VOD. Studies analyzing endothelial protection against further conditioning agents should help to clarify whether Defibrotide can be used as a broad protective agent.

References

1. Holler E, Kolb H J, Möller A et al. Increased serum levels of tumor necrosis factor a precede major complications of bone marrow transplantation. Blood. 1990;75:1011-1016.
2. Antin J H, Ferrara J L M. Cytokine dysregulation and acute graft-versus-host disease. Blood. 1992;80:2964-2968.
3. Ferrara J L, Levy R, Chao N J. Pathophysiological mechanisms of acute graft-vs.-host disease. Biol Blood Marrow Transplant. 1999;5:347-356.
4. Xun C Q, Brown B A, Jennings C D, Henslee-Downey P J, Thompson J S. Acute graft-versus-host-like diseases induced by transplantation of human activated natural killer cells into SCID mice. Transplantation. 1993;56:409-417.
5. Weiner R S, Bortin M M, Gale R P et al. Interstitial pneumonitis after bone marrow transplantation. Assessment of risk factors. Ann Intern Med. 1986;1 04:168-175.
6. Weiss M A. Novel treatment strategies in chronic lymphocytic leukemia. Curr On-col Rep. 2001 ;3:217-222.
7. Wasch R, Reisser S, Hahn J et al. Rapid achievement of complete donor chimerism and low regimen-related toxicity after reduced conditioning with fludarabine, carmustine, melphalan and allogeneic transplantation. Bone Marrow Transplant. 2000;26:243-250.
8. Carella A M, Champlin R, Slavin S, McSweeney P, Storb R. Mini-allografts: ongoing trials in humans. Bone Marrow Transplant. 2000;25:345-350.
9. Slavin S, Nagler A, Naparstek E et al. Nonmyeloablative stem cell transplantation and cell therapy as an alternative to conventional bone marror transplantation with lethal cytoreduction for the treatment of malignant and nonmalignant hematologic diseases. Blood. 1998;91 :756-763.
10. Holler E, Kolb H J, Hiller E et al. Microangiopathy in patients on cyclosporine prophylaxis who developed acute graft-versus-host disease after HLA-identical bone marrow transplantation. Blood. 1989;73:2018-2024.
11. Eissner G , Kohlhuber F, Grell M et al. Critical involvement of transmembrane TNF-α in endothelial programmed cell death mediated by ionizing radiation and bacterial endotoxin. Blood. 1995;86:4184-4193.
12. Lindner H, Holler E., Ertl B et al. Peripheral blood mononuclear cells induce programmed cell death in human endothelial cells and may prevent repair-role of cytokines. Blood. 1997;89:1931-1938.
13. Haimovitz-Friedman A, Balaban N, Mcloughlin M et al. Protein kinase C mediates basic fibroblast growth factor protection of endothelial cells against radiation-induced apoptosis. Cancer Res. 1994;54:2591-2597.
14. Fuks Z, Persaud R S, Alfieri A et al. Bacic fibroblast growth factor protects endothelial cells against radiation-induced programmed cell death in vitro and in vivo. Cancer Res. 1994;54:2582-2590.
15. Eissner G, Lindner H, Behrends U et al. Influence of bacterial endotoxin on radiation-induced activation of human endothelial cells in vitro and in vivo, protective role of IL-10. Transplantation. 1996;62:819-827.
16. Lindner H, Holler E, Gerbitz A, Johnson J P, Bornkamm G W, Eissner G. Influence of bacterial endotoxin on radiation-induced activation of human endothelial cells in vitro and in vivo: 2. IL-10 protects against transendothelial migration. Transplantation. 1997;64:1370-1973.
17. Beelen D W, Haralambie E, Brandt H et al. Evidence that sustained growth suppression of intestinal anaerobic bacteria reduces the risk od acute graft-versus-host disease after sibling marrow transplantation. Blood. 1992;80: 2668-2676.
18. Eissner G, Multhoff G, Holler E. Influence of bacterial endotoxin on the allogenicity of human endothelial cells. Bone Marrow Transplant. 1998;21:1286-1287.
19. Ades E W, Candal F J, Swerlick R A et al. HMEC-1: establishment of an immortalized human microvascular endothelial cell line. J Invest Dermatol. 1992;99:683-690.
20. Cotter T G, Lennon S V, Glynn J M, Green D R. Microfilament-disrupting agents prevent the formation of apoptotic bodies in tumor cells undergoing apoptosis. Cancer Res. 1992;52:997-1005.
21. Lee A, Whyte M K, Haslett C. Inhibition of apoptosis and prolongation of neutrophil functional longevity by inflammatory mediators. J Leukoc Biol. 1993;54:283-288.
22. Westphal J R, Tax W J, Willems H W, Koene R A, Ruiter D J, De-Waal RM. Accessory function of endothelial cells in anti-CD3-induced T-cell proliferation: synergism with monocytes. Scand J Immunol. 1992;35:449-457.
23. MacDonald H R, Engers H D, Cerrottini J C, Brunner K T. Generation of cytotoxic T lymphocytes in vitro. J Exp Med. 1974;140:718-722.
24. Long E. Regulation of immune responses through inhibitory receptors. Annu Rev Immunol. 1999;17:875-904.
25. Nagler A, Aker M, Or R et al. Low-intensity conditioning is sufficient to ensure engraftment in matched unrelated bone marrow transplantation. Exp Hematol. 2001 ;29:362-370.
26. Michallet M, Bilger K, Garban F et al. Allogeneic hematopoietic stem-cell tranplantation after nonmyeloablative preparative regimens: impact of pretransplantation and posttransplantation factors on outcome. J Clin Oncol. 2001;19:3340-3349.
27. Holler E, personal communication
28. Hildebrandt G, Bertz H, Mestan A et al. Analysis of pulmonary function after allogeneic bone marrow trasplantation (BMT) or blood stem cell transplantation (PBSCT) using conditioning regimens with total body irradiation (TBI) and conventional intensity compared to regimens without TBI with reduced intensity [abstract]. Bone Marrow Transplant. 2001;27 (Suppl. 1):S216.
29. Bornhauser M, Thiede C, Schuler U et al. Dose-reduced conditioning for allogeneic blood stem cell transplantation: durable engraftment without antithymocyte globulin. Bone Marrow Transplant. 2000;26:119-125.
30. Huang P, Siciliano M J, Plunkett W. Gene deletion, a mechanism of induced mutation by arabinosyl nucleosides. Mutat Res. 1989;210:291-301.
31. Genini D, Budihardjo I, Plunkett W et al. Nucleotide requirements for the in vitro activation of the apoptosis protein-activating factor-1-mediated caspase pathway. J Biol Chem. 2000;275:29-34.
32. Biederman B C, Pober J S. human vascular endothelial cells favor clonal expansion of unusual alloreactive CTL. J Immunol. 1999;162:7022-7030.
33. Briscoe D M, Alexander A I, Lichtman A H. Interactions between T lymphocytes and endothelial cells in allograft rejection. Curr Op Immunol. 1998;1 0:525-531.
34. Pegram M, Kennedy L D. Prevention and treatemnt of veno-occlusive disease. Ann Pharmacother. 2001;35:935-942.
35. Rossini G, Pompilio G, Biglioli P et al. Protectant activity of defibrotide in cardioplegia followed by idchemia/(repersusion injury in the isolated rat heart. J Card Surg. 1999;14:334-341.
36. Rossini G, Berti F, Trento F et al. Defibrotide normalizes cardiovascular function hampered by established atherosclerosis in the rabbit. Thromb Res. 2000;97:29-38.
37. Pogliani E M, Perseghin P, Parma M, Pioltelli P, Corneo G. Defibrotide in recurrent thrombotic thrombocytopenic purpura. Clin Appl Thromb Hemost. 2000;6:69-70.
38. San T, Moini H, Emerk K, Bilsel S. Protective effect of defibrotide on perfusion induced endothelial damage. Throm Res. 2000;99:335-341.
39. Chopra R., Eaton J D, Grassi A et al. Defibrotide for the treatment of hepatic veno-occlusive disease: results of the European compassionate-use study. Br J Haematol. 2000;111:1122-1129.

1. A method of treating a patient undergoing treatment with an immunosuppressant comprising a step of administering to the patient a therapeutically effective dose of a protective oligodeoxyribonucleotide and achieving a reduction in complications related to treatment with an immunosuppressant.

2. A method of treating a patient undergoing treatment with an immunosuppressant comprising a step of administering to the patient a therapeutically effective dose of a protective oligodeoxyribonucleotide and achieving protection of one or both of the patient's epithelial or endothelial cells from the effects of the immunosuppressant.

3. A method of treating a patient undergoing treatment with an immunosuppressant comprising a step of administering to the patient a therapeutically effective dose of a protective oligodeoxyribonucleotide for and achieving protection one or both of the patient's epithelial or endothelial cells from one or both of apoptosis or activation induced by the administration of the immunosuppressant.

4. The method according to claim 1 wherein the immunosuppressant is a nucleoside.

5. The method according to claim 1 wherein the immunosuppressant is chosen from the group comprising 5-fluorouracil, methotrexate, fludarabine, vincristine, vinblastine, paclitaxel, docetaxel, cyclophosphamide, bischloroethylnitrosurea, melphalan, cisplatin, carboplatin, oxaliplatin, JM-216. Ci-973, doxorubicin, daunorubicin, mitomycin-C, etoposide, camptothecin, cyclosporin, tacrolimus, sirolimus, or combinations thereof.

6. (canceled)

7. The method according to claim 1 wherein the protective oligodeoxyribonucleotide is defibrotide.

8. The method according to claim 1 wherein the step of administering the protective oligodeoxyribonucleotide occurs as one or more of concurrently with, concomitantly with, simultaneously with, after, or before the administration of the immunosuppressant to the patient.

9. The method according to claim 1 wherein the step of administering the protective oligodeoxyribonucleotide occurs after that of administering the immunosuppressant to the patient.

10. The method according to claim 9 wherein the time delay between the step of administering the protective oligodeoxyribonucleotide and that of administering the immunosuppressant to the patient is from about one hour to about two weeks.

11. The method according to claim 1 wherein the step of administering the protective oligodeoxyribonucleotide occurs before that of administering the immunosuppressant to the patient.

12. The method according to claim 11 wherein the time difference between the step of administering the protective oligodeoxyribonucleotide and that of administering the immunosuppressant to the patient is from about one hour to about two weeks.

13. The method according to claim 7 wherein the dose of the defibrotide administered is chosen so as to reach a blood level in the patient from about 100 μg/mL to about 0.1 μg/mL.

14. The method according to claim 13 wherein the dose of defibrotide administered is chosen so as to reach a blood level in the patient of about 10 μg/mL.

15. The method according claim 7 wherein the dose of defibrotide administered is from about 100 mg/kg body weight of the patient to about 0.01 mg/kg body weight.

16. The method according to claim 15 wherein the dose of defibrotide administered is from about 15 mg/kg body weight of the patient to about 1 mg/kg body weight.

17. The method according to claim 3 wherein the activation includes enhanced expression of ICAM-1.

18. The method according to claim 1 wherein the treatment with an immunosuppressant occurs during stem cell transplantation.

19. The method according to claim 18 wherein the stem cell transplantation is allogeneic stem cell transplantation.

20. A pharmaceutical composition containing a therapeutically effective dose of an immunosuppressant and of a protective oligodeoxyribonucleotide.

21. A pharmaceutical composition according to claim 20 constituted by two different separately administrable formulations, one formulation containing the immunosuppressant and the other formulation containing the protective oligodeoxyribonucleotide.

22. A pharmaceutical composition according to claim 20 as a combined preparation for one or more of simultaneous, separate, or sequential administration.

23. A pharmaceutical composition according to claim 20 wherein the immunosuppressant is a nucleoside.

24. A pharmaceutical composition according to claim 20 wherein the immunosuppressant is chosen from the group comprising 5-fluorouracil, methotrexate, fludarabine, vincristine, vinblastine, paclitaxel, docetaxel, cyclophosphamide, bischloroethylnitrosurea, melphalan, cisplatin, carboplatin, oxaliplatin, JM-2 16, Ci-973, doxorubicin, daunorubicin, mitomycin-C, etoposide, camptothecin, cyclosporin, tacrolimus, sirolimus, or combinations thereof.

25. (canceled)

26. A pharmaceutical composition according to claim 20 wherein the protective oligodeoxyribonucleotide is defibrotide.

27. A pharmaceutical composition according to claim 20 characterized by further containing one or more of customary excipients or adjuvants.

28. A pharmaceutical composition according to claim 20 characterized in that the composition is intravenously injectable.

29. The method according to claim 9 wherein the time delay between the step of administering the protective oligodeoxyribonucleotide and that of administering the immunosuppressant to the patient is from about two days to about seven days.

30. The method according to claim 11 wherein the time difference between the step of administering the protective oligodeoxyribonucleotide and that of administering the immunosuppressant to the patient is from about two hours to about two days.

31. The method according to claim 7 wherein the dose of the defibrotide administered is chosen so as to reach a blood level in the patient from about 10 μg/mL to about 100 μg/mL.

32. The method according claim 7 wherein the dose of defibrotide administered is from about 20 mg/kg weight of the patient to about 0.1 mg/kg body weight.

33. The method according claim 32 wherein the dose of defibrotide administered is about 12 mg/kg weight of the patient.

34. A pharmaceutical composition according to claim 20 as a combined preparation for separate administration.

35. A pharmaceutical composition according to claim 20 as a combined preparation for sequential administration.

36. The method according to claim 1 wherein the immunosuppressant is chosen from the group comprising antimetabolites, anti-microtubule agents, taxanes, alkylating agents, platinum agents, anthracyclines, antibiotic agents, topoisomerase inhibitors, other cytotoxic agents, or combinations thereof.

37. A pharmaceutical composition according to claim 20 wherein the immunosuppressant is chosen from the group comprising antimetabolites, anti-microtubule agents, taxanes, alkylating agents, platinum agents, anthracyclines, antibiotic agents, topoisomerase inhibitors, other cytotoxic agents, or combinations thereof.

38. The method according to claim 1 wherein the treatment with an immunosuppressant occurs during bone marrow transplantation.
